SEQUENCE LISTING (6 sequences)

SEQ ID NO: 1 - protein, 343 aa, Influenza A virus
MNTQILAFALLGIAVTNADKICLGHHAVSNGTKVNTLTEKGVEVVNATETVESTSLKDICGGGGCEGECFYSGGTINSKLPFQNIDSRAVGKCPRYVKQKSLLLATGMKNVPEVAKRKRQARGLFGAIAGFIENGWEGLIDGWYGFRHQNAQGEGTAADYKSTQAAIDQITGKLNRLIEKTNQQFELIDNEFSEVEQQIGNVINWTRDSITEIWSYNAELLVAMENQHTIDLADSEMNKLYERVRRQLRENAEEDGTGCFEIFHKCDDNCMESIRNNTYDHTQYREEALQNRIQIDPVKLSSGYKDVILWFSFGASCFVLLAIVMGLVFICVKNGNMRCTICI

SEQ ID NO: 2 - protein, 355 aa, Influenza A virus
MLSIIVLSLILAETSAQKISNNDTGNPVICLGHHAVPNGTIVKTLTNDQIEVVSAQELVESSNIDELCGGGGCTSECITDKGSISSDKPFQNVSRIAIGDCPKYVKQSSLKLATGMRNIPEKQTRGLFGAIAGFIENGWQGLIDGWYGFRHQNAEGTGTAADLKSTQAAIDQINGKLNRLIEKTNEKYHQIEKEFEQVEGRIQDLEKYVEDTKIDLWSYNAELLVALENQHTIDVTDSEMNKLFERVRRQLRENAEDQGNGCFEIFHKCDNNCIESIRNGTYDHNIYRDEAINNRFQIKGVELTQGYKDVILWISFSISCFLLVALLLGFILWACQNGNIRCQICIVHLKTPLFL

SEQ ID NO: 3 - protein, 347 aa, Influenza A virus
MMMALVILLLLFTTVKSDQICIGYHANNSTEKVDTILEKNVTVTHSVNLLEKTHNGKLCGGGGCNTTCQTPEGAINTSLPFHNVHPLTIGECPKYVKSEKLRLATGLRNVPQIESRRKRGLFGAIAGFIEGGWTGMVDGWYGYHHSNEQGSGYAADKESTQKAIDGITNKVNSVIDKMNTQFEAVGKEFNNLERRIENLNKKMEDGFLDVWTYNAELLVLMENERTLDFHDSNVKNLYDKVRMQLRDNAKELGNGCFEFYKCDNECMESVKNGTYDYPKYSEEAKLNREKIEGVKLESMGVYQILAIYSTVASSLVLVVMIAGISFWMCSNGSLQCRICIICELRLL

SEQ ID NO: 4 - protein, 339 aa, Influenza A virus
MEKFILISILLAATVANYDKICIGYQSNNSTETVNTLIEQNVPVTQAKELVHTEKNPVLCGGGGCTTQCQTEKGALNTSKPFQNVSKHYIGKCPKYIPSASLKLAVGLRNVPSIQSRGLFGAIAGFIEGGWSGLVAGWYGFQHSNSEGTGMAADRDSTQKAIDKITNKVNNIVDKMNKQFEVVDHEFSEVESRINMINNKIDDQIQDIWAYNAELLVLLENQKTLDEHDANVRNLYDKVKRALSSNAIDTGNGCFEILHKCDNQCMETIRNGTYNHREYEEESKLERQKINGVKLEENSTYKILSIYSTVASSLVLALMIAGGLIFGMQNGSCRCTFCI

SEQ ID NO: 5 - protein, 340 aa, Influenza A virus
MEVKLLFILIIVYGVKADKICIGYLSNNSTDKVDTLIENNVPVTSSVDLVETNHTGTYCGGGGCNTKCQTSVGGINTNKTFQNIERNAIGDCPKYIKSGQLKLATGLRNVPSIASRGLFGAIAGFFIEGGWPGLINGWYGFQHQNEQGTGIAADKESTQKAIDQITTKINNIIEKMNGNYDSIRGEFNQVEERINMLADRVDDAVTDVWSYNAKLLVLLENDKTLDLHDANVRNLHDQVRRALKNNAIDEGNGCFNLLHKCNDSCMETIRNGTYNHKEYEEESKLKRQEIEGIKLKSEDNVYKILSIYSCIASSVVLVGLILAFIMWACSSGNCRFNICI

SEQ ID NO: 6 - protein, 339 aa, Influenza A virus
MELIVLLILLNPYTFVLGDRICIGYQANQNNQTVNTLLEQNVPVTGAQEILETNHNGKLCGGGGCSTKCQTPLGALNSTLPFQNVHQQTIGNCPKYVKATSLMLATGLRNNPQMEGRGLFGAIAGFIEGGWQGMIDGWYGYHHENQEGSGYAADKEATQKAVDAITNKVNSIIDKMNSQFESNIKEFNRLELRIQHLSDRVDDALLDIWSYNTELLVLLENERTLDFHDANVKNLFEKVKAQLKDNAIDEGNGCFLLLHKCNNSCMDDIKNGTYKYMDYREESHIEKQKIDGVKLTDYSRYYIMTLYSTIASSVVLGSLIIAAFLWGCQKGSIQCKICI

(19) United States
(12) Patent Application Publication
(10) Pub. No.: US 2024/0000916 A1
(43) Pub. Date: Jan. 4, 2024

(54) DESIGN OF OPTIMIZED UNIVERSAL INFLUENZA VACCINES, THEIR DESIGNS AND USES

(71) Applicant: Greffex, Inc., Houston, TX (US)

(72) Inventors: Uwe D. STAERZ, Denver, CO (US); Philip C. SPEAR, Aurora, CO (US); Yan QI, Lone Tree, CO (US); Janae Wheeler CULL, Brighton, CO (US)

(21) Appl. No.: 18/252,790

(22) PCT Filed: Nov. 18, 2021
(86) PCT No.: PCT/US2021/059915

§ 371 Date: May 12, 2023

Related U.S. Application Data
(60) Provisional application No. 63/115,459, filed on Nov. 18, 2020.

(51) Int. Cl. A61K 39/145; A61P 31/16

(52) U.S. Cl. CPC A61K 39/145; A61P 31/16

(57) ABSTRACT

The present disclosure provides a universal influenza virus vaccine. A composition for a universal influenza virus vaccine comprises at least two, preferably more than two, different influenza hemagglutinin (HA) derived antigens. The HA proteins from which the antigens are derived have a hypervariable region located between conserved cysteines at positions 52 and 277, and the hypervariable region is deleted in the antigens. The at least two antigens each have a similarity with HA molecules of more than one influenza serotype in excess of 60, or 70, or 80, as calculated by the emboss explorer cons program.

### CROSS REFERENCE TO RELATED APPLICATIONS

This application claims priority to and is a non-provisional application of Provisional Application No. 63/115,459, filed on Nov. 18, 2020, which is incorporated herein by reference in its entirety.

### SUBMISSION OF SEQUENCE LISTING

A sequence listing containing the file named Sequence_Listing which is 18 kilobytes in size (measured in MS-Windows) and was created on Nov. 18, 2021, is provided herewith and is incorporated by reference in its entirety.

### FIELD

The disclosure relates to compositions containing influenza HA derived antigens which have high similarity to more than one influenza A serotype. In another embodiment, the disclosure relates to compositions for a universal influenza vaccine.

### BACKGROUND

Seasonal influenzas, pandemic influenzas and highly pathological avian influenzas (HPAI) arise from large reservoirs of birds and mammals. New influenza variants can quickly recombine in unpredictable permutations. Since minor genetic changes of an HPAI may greatly increase its infectivity and allow efficient human-to-human infections, pandemic spreads may indeed become reality.

For instance, the H5N1 HPAI erupted in Asia in 2003 through bird-to-human infections. It spread throughout the world with Egypt being its hotspot. The original H5N1 HPAI strain of Asia was of clade 1, whereas the more recent Egyptian strain is of clade 2 (clade 2.2.1). The first H7N9 HPAI appeared in China in 2013, followed by further variants in 2016 and 2017. The U.S. Federal government has maintained stockpiles of egg-based conventional H5N1 and H7N9 vaccines. Their immunogenicities, especially of the Sanofi-Pasteur clade 1 H5N1 vaccine, were significantly lower than those of standard seasonal influenza vaccines. The vaccines are given in a prime-boost regimen, and the clade 2 H5N1 GSK vaccine is enhanced by an adjuvant. Other issues are both the reduced level of heterologous protection between the clade 1 and clade 2 H5N1 vaccines, and also the transient nature of the immune response, which may become ineffective within 6 months.

It has been proposed to deal with the unpredictable nature of the influenza virus with broadly reactive vaccines so-called “universal” influenza vaccines. The most potent anti-influenza antibodies (Ab) bind to the hemagglutinin (HA) head and physically block the interaction with the specific cell receptor. The high variability of this HA domain provides specificity, but limits cross-reactivity. Much rarer Abs recognize the highly conserved HA stem regions. They bind broadly to several influenza A subtypes. Stem antibodies may not act by inhibiting cell receptor binding, but rather by inhibiting virus-membrane fusion, including ADCC of infected cells and preventing viral entry into the nucleus. Current two-antigen stem vaccines may, however, deliver only a base immunity. Such two-antigen stem vaccines are based to two different “conserved” antigens that only have relatively low similarity or “sequence-overlap” with HAs from influenzas of the different serotypes. It may therefore be necessary to boost the immune responses induced by the two-molecule universal antigens with a vaccine based on a specific influenza strain. Some scientific evidence also suggests that broadly reactive Abs could have issues, such as, the induction of disease-enhancing Abs that aggravate subsequent influenza infections.

This present approach to immune prophylaxis against seasonal and pandemic influenzas as well as HPAIs (as well as other newly emerging infectious threats) is severely lacking, as the following issues have not been adequately addressed in existing solutions:
(1) Potency: The present influenza vaccines including the stockpiled HPAI vaccines provide only modest and transient immune protection.
(2) Development Speed: The present development and production schemes for influenza vaccines are slow and do not meet the challenges of fast emerging influenzas (and other infectious threats).
(3) Variability of Influenza Strains: Influenzas have the ability to quickly alter their antigenic composition, and new vaccines need to be constantly developed to account for new strains and address new specificities.

It is desirable to develop a more consistent “universal” vaccine that uses conserved antigens derived from the influenza hemagglutinins, maintains high similarity or conservation with the proteins found in influenzas of different serotypes, and induces potent immune responses.

Classical virus vaccines are produced as attenuated, inactivated viruses or virus extracts using slow and capacity limited fertilized egg technologies. More recent tissue culture approaches include viral expansion and synthesis of specific antigens in cell culture broth in the case of influenza and also ZIKV (zika virus) vaccines. The intrinsically low immunogenicity of protein-based vaccines can and has to be enhanced by the addition of adjuvants.

Rather than delivering a completed vaccine, genetic immunization approaches force the host to synthesize and present antigens to the immune system. These systems more closely mimic natural viral infections. Different strategies are being investigated. Naked DNA vaccines, while simple and quick to produce, require relatively high doses and specialized delivery systems to overcome their low immunogenicity. Genetic vaccines often take advantage of engineered viruses, such as Ad, vaccinia virus, vesicular stomatitis virus, yellow fever virus, and alpha viruses. However, their underlying biology may limit their utility. They may be pathogenic as, for instance, the 17D yellow fever virus strain, which has been linked to severe, albeit rare, adverse reactions.

Besides naked DNA, AAV and also RNA-viruses as the foundation of a gene transfer and also vaccine vector platform, adenovirus (Ad)-based approaches are available. Ad-derived vectors have proven benign, avoid integration into the host genome, and are intrinsically adjuvanted. Numerous vaccines have been engineered on the basis of replication-deficient, minimally modified early generation (eg) Ad vectors. They have repeatedly demonstrated higher immunogenicity in head-to-head comparisons with other vaccine systems. Furthermore, it has been demonstrated that they also raised potent immune responses to avian influenzas and the ZIKV. Importantly and in contrast to other vaccine systems, they deliver sustained immune protection over extended periods of time. Therefore, they have seen renewed interest with vaccines against ZIKV, Ebola, tuberculosis and malaria.

Ad vector systems were developed that integrated different strategies to overcome the limitations of earlier Ad systems. Fully deleted (fd) Ad vector platforms package vaccine genomes into human serotypes of low prevalence, such as the human serotype Ad6. Such vaccines are fully deleted (fd) of all endogenous Ad genes. fdAd vectors better focus the immune system to a vaccine antigen, minimize interference by anti-Ad immune responses, and enable prime-boost vaccination. fdAd vector systems that use helper viruses for encapsidation are linked to contaminations with helper viruses and replication competent adenovirus (RCA). These impurities have the potential to induce potent anti-Ad responses. A new fdAd architecture was produced—fdAd vectors that packaged fdAd independently of a helper virus (helper virus-independent, hi). These are fully deleted helper virus-independent Ad vectors (fdhiAd vectors).

A fdhiAd vaccine platform is built upon two independently modifiable components: (i) fdAd vector genome modules deleted of all endogenous Ad genes, and (ii) non-packageable circular packaging expression plasmids that deliver the necessary Ad late genes in trans.

To obtain fdAd vector genomes deleted of all endogenous Ad genes, the fdAd vector base modules are assembled to accommodate different transgene constructs of up to 33 kb. They carry the left and right ITRs and packaging signals (Ψ) of different Ad.

The completed fdAd vector genome modules are about 34 kb in size and are encapsidated by co-transfection of a non-packageable packaging expression plasmid into host cells. The packaging expression plasmids provide in trans all Ad genes necessary of the assembly of the capsid, replication of the fdAd vector genome module and its integration into the capsid. Different circular packaging expression plasmids have been engineered on a modified pBR322 backbone for capsids of the human Ad species C and B (serotype 35). They provide the crucial late genes (L1, L2, L3, L4, L5) together with the early genes E2 and E4 in trans and are deleted of the packaging signal Ψ and at least one ITR.

fdAd technologies have a large payload, which can be exploited to deliver large transgene constructs. For instance, it had been possible to deliver the full-size human coagulation factor VIII cDNA together with the immune suppressive gene CD8 (combined 12 kb) in a single fdAd vector. Both transgenes were efficiently expressed upon transduction into cells. Therefore, fdAd vectors can be used as basis for the production of an optimized universal influenza vaccine that delivers more than two conserved influenza antigen constructs.

Present universal influenza vaccines are built upon two conserved influenza hemagglutinin constructs. The design of the initial universal influenza vaccines followed the reactivity pattern of two antibodies, CR6261 and CR8020, that bind to epitopes in the alpha helical structure in the stem region. Antibodies against this region neutralize viral particles. It appears these antibodies inhibit HA function by preventing full length HA from undergoing pH induced conformational changes necessary to fuse to membranes. CR6261 and CR8020 neutralize broad ranges of HA targets as these are heavily conserved regions due to their function. CR6261 was shown to neutralize HA in group 1, subtypes 1, 2, 5, 6, 9, 13 and 16. CR8020 was shown to bind to group two HAs” 3, 4, 7, 10, 14 and 15. Designing two conserved hemagglutinin antigens that followed these two groups lead to an average sequence identity of approximately 55%. Even though headless constructs are a promising approach to a universal flu vaccine, technical issues have prevented a broader development of this approach. The low identity score of a two antigen construct approach lead to low affinity immune responses. Furthermore, increasing the number of antigen constructs to overcome the low sequence overlap made the approach difficult and expensive. Headless HAs are not capable of forming viable influenza viruses which can propagate in egg cultures to be inactivated for vaccines. Therefore they have to be produced by alternative means.

fdAd vector systems overcome these production issues. The headless HAs do not need to function in influenza viruses or virus like particles, they just need to be expressed in transduced cell in vivo. Additionally, fdAd can deliver more than two HA constructs with ease to their large payload. The multivalency of an fdAd vaccine does not significantly increase the complexity of engineering or production. Therefore a multivalent universal influenza vaccine can be produced cost effectively.

### SUMMARY

In an embodiment the present disclosure provides a composition for a vaccine. In accordance with embodiments of the present disclosure, a composition for a vaccine comprises at least two different influenza hemagglutinin (HA) derived antigens, wherein an HA protein from which the antigens are derived includes a hypervariable region and the hypervariable region is deleted from the at least two different influenza HA derived antigens, and wherein each of the at least two different influenza HA derived antigens has a similarity with HA molecules of more than one influenza serotype in excess of at least 60, as calculated by the emboss explorer cons program.

In an embodiment, the similarity is at least 70, or at least 80.

In an embodiment, the hypervariable region is replaced by a peptide linker in the at least two different influenza HA derived antigens.

In an embodiment, the at least two different influenza HA derived antigens are proteins. In another embodiment, the at least two different influenza HA derived antigens are one of RNA and DNA coding for the HA protein from which the antigens are derived. In a further embodiment, the one of RNA and DNA are in a viral vector.

In an embodiment, the hypervariable region is located between conserved cysteines at positions 52 and 277 using the amino acid numbering of an influenza HA of the serotype H3.

In another embodiment, the composition comprises more than two different influenza HA derived antigens.

In an embodiment the present disclosure provides a method for producing a universal influenza virus vaccine. In accordance with embodiments of the present disclosure, a method for producing a universal influenza virus vaccine comprise obtaining a first influenza HA derived antigen having a similarity with HA molecules of a first plurality of influenza serotypes in excess of at least 60, as calculated by the emboss explorer cons program; and obtaining a second influenza HA derived antigen having a similarity with HA molecules of a second plurality of influenza serotypes in excess of at least 60, as calculated by the emboss explorer cons program, wherein the first and second plurality of influenza serotypes are composed of different serotypes.

In an embodiment, the first and second plurality of influenza serotypes are composed of different and non-overlapping serotypes.

In an embodiment, the HA proteins from which the first and second influenza HA derived antigens are derived contains a hypervariable region, and the hypervariable region is deleted from the first and second influenza HA derived antigens.

In an embodiment, the similarities are, independently, at least 70, or at least 80.

In an embodiment, the hypervariable region is replaced by a peptide linker.

In an embodiment, the first and second influenza HA derived antigens are proteins. In another embodiment, the first and second influenza HA derived antigens are one of RNA and DNA coding for the HA protein from which the antigens are derived. In a further embodiment, the method comprises encapsidating the one of the RNA and DNA coding the HA protein into a viral vector.

In an embodiment, the method comprises obtaining at least a third influenza HA derived antigen having a similarity with HA molecules of a third plurality of influenza serotypes in excess of at least 60, as calculated by the emboss explorer cons program.

In an embodiment the present disclosure provides a method of vaccinating an animal against at least two different influenza serotypes. In accordance with embodiments of the present disclosure, the method comprises providing a vaccine composition comprising at least two different influenza hemagglutinin (HA) derived antigens, wherein an HA protein from which the antigens are derived includes a hypervariable region and the hypervariable region is deleted from the at least two different influenza HA derived antigens, and wherein each of the at least two different influenza HA derived antigens has a similarity with HA molecules of more than one influenza serotype in excess of at least 60, as calculated by the emboss explorer cons program; and delivering the vaccine composition to the animal.

### BRIEF DESCRIPTION OF THE SEQUENCE LISTING

SEQ ID NO. 1: shows the consensus sequence for the red influenza serotypes as shown in FIG. 1.

SEQ ID NO. 2: shows the consensus sequence for the orange influenza serotypes as shown in FIG. 1.

SEQ ID NO. 3: shows the consensus sequence for the yellow influenza serotypes as shown in FIG. 1.

SEQ ID NO. 4: shows the consensus sequence for the green influenza serotypes as shown in FIG. 1.

SEQ ID NO. 5: shows the consensus sequence for the blue influenza serotypes as shown in FIG. 1.

SEQ ID NO. 6: shows the consensus sequence for the purple influenza serotypes as shown in FIG. 1.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a representation of sequence identity of representative influenza A hemagglutinin proteins grouped into six homology groups.

FIG. 2 is a representation of a hemagglutinin protein phylogenetic tree.

FIG. 3 is a representation of conserved hemagglutinin stem transgene constructs as protein sequences.

FIG. 4 is a representation of sequence similarities of representative influenza A hemagglutinin proteins with the six consensus conserved stem proteins.

FIG. 5 is a diagrammatic representation of a fully deleted helper virus-independent adenoviral vector platform.

### DETAILED DESCRIPTION

Before any embodiments of the present disclosure are explained in detail, it is to be understood that the disclosure is not limited in its application to the details of construction and the arrangement of components set forth in the following description or illustrated in the drawings. The disclosure is capable of other embodiments and of being practiced or of being carried out in various ways. Also, it is understood that the phraseology and terminology used herein is for the purpose of description and should not be regarded as limiting. The use of “including,” “comprising” or “having” and variations thereof herein is meant to encompass the items listed thereafter and equivalents thereof as well as additional items. The use of “including essentially” and “consisting essentially of” and variations thereof herein is meant to encompass the items listed thereafter, as well as equivalents and additional items provided such equivalents and additional items to not essentially change the properties, use or manufacture of the whole. The use of “consisting of” and variations thereof herein is meant to include the items listed thereafter and only those items.

With reference to the drawings, like numbers refer to like elements throughout. It will be understood that, although the terms first, second, etc., may be used herein to describe various elements, components, regions, and/or sections, these elements, components, regions and/or sections should not be limited by these terms. These terms are used only to distinguish one element, component, region and/or section from another element, component, region and/or section. Thus, a first element, component, region or section could be termed a second element, component, region or section without departing from the disclosure.

The numerical ranges in this disclosure are approximate, and thus may include values outside of the range unless otherwise indicated. Numerical ranges include all values from and including the lower and the upper values (unless specifically stated otherwise), in increments of one unit, provided that there is a separation of at least two units between any lower value and any higher value. As an example, if a compositional, physical or other property, such as, for example, amount of a component by weight, etc., is from 10 to 100, it is intended that all individual values, such as 10, 11, 12, etc., and sub ranges, such as 10 to 44, 55 to 70, 97 to 100, etc., are expressly enumerated. For ranges containing explicit values (e.g., a range from 1, or 2, or 3 to 5, or 6, or 7), any subrange between any two explicit values is included (e.g., the range 1-7 above includes subranges 1 to 2; 2 to 6; 5 to 7; 3 to 7; 5 to 6, etc.). For ranges containing values which are less than one or containing fractional numbers greater than one (e.g., 1.1, 1.5, etc.), one unit is considered to be 0.0001, 0.001, 0.01 or 0.1. as appropriate. For ranges containing single digit numbers less than ten (e.g., 1 to 5), one unit is typically considered to be 0.1. These are only examples of what is specifically intended, and all possible combinations of numerical values between the lowest value and the highest value enumerated, are to be considered to be expressly stated in this disclosure.

Spatial terms, such as “beneath,” “below,” “lower,” “above,” “upper,” and the like, may be used herein for ease of description to describe one element's or feature's relationship to another element(s) or feature(s) as illustrated in the figures. It will be understood that the spatially relative terms are intended to encompass different orientations depending on the orientation in use or illustration. For example, if a device in the figures is turned over, elements described as “below” or “beneath” other elements or features would then be oriented “above” the other elements or features. Thus, the exemplary term “below” can encompass both an orientation of above and below. A device may be otherwise oriented (rotated 90° or at other orientations) and the spatially relative descriptors used herein interpreted accordingly.

As used herein, the term “and/or” includes any and all combinations of one or more of the associated listed items. For example, when used in a phrase such as “A and/or B,” the phrase “and/or” is intended to include both A and B, A or B; A (alone); and B (alone). Likewise, the term “and/or” as used in a phrase such as “A, B and/or C” is intended to encompass each of the following embodiments” A, B and C; A, B, or C; A or C; A or B or C; A and C; A and B; B and C; A (alone); B (alone); and C (alone).

Unless defined otherwise, all technical and scientific terms used herein have the same meaning as commonly understood by one of ordinary skill in the art to which this disclosure belongs. Generally, the nomenclature used herein and the laboratory procedures in cell culture, molecular genetics, and nucleic acid chemistry and hybridization described below are those well-known and commonly employed in the art. Standard techniques are used for recombinant nucleic acid methods, polynucleotide synthesis, and microbial culture and transformation (e.g., electroporation, lipofection). Generally, enzymatic reactions and purification steps are performed according to the manufacturer's specifications. The techniques and procedures are generally performed according to conventional methods in the art and various general references (see generally, Sambrook et al. Molecular Cloning: a Laboratory Manual, 2d ed. (1989) Cold Spring Harbor Laboratory Press, Cold Spring Harbor. N.Y., which is incorporated herein by reference), which are provided throughout this document. Units, prefixes, and symbols may be denoted in their S1 accepted form. Unless otherwise indicated, nucleic acids are written left to right in 5′ to 3′ orientation; amino acid sequences are written left to right in amino to carboxyl orientation, respectively. Amino acids may be referred to herein by either their commonly known three letter symbols or by the one-letter symbols recommended by the IUPAC-IUB Biochemical nomenclature Commission. Nucleotides, likewise, may be referred to by their commonly accepted single-letter codes. Unless otherwise provided for, software, electrical, and electronics terms as used herein are as defined in The New IEEE Standard Dictionary of Electrical and Electronics Terms (5.sup.th edition, 1993).

The embodiments disclosed herein relate to the design, construction and production of multivalent universal influenza A vaccines.

As employed throughout the disclosure, the following terms, unless otherwise indicated, shall be understood to have the following meanings and are more fully defined by reference to the specification as a whole.

The terms “adenovirus,” “adenovirus virion,” and “adenovirus particle” as used herein include any and all viruses that may be categorized as an adenovirus, including any adenovirus that infects a human or an animal, including all groups, subgroups, species and serotypes.

The term “adenovirus vector” as used herein includes any genetic construct or viral constructs that are based on an adenovirus and used to transfer genetic material. The terms “deleted adenovirus” or “deleted adenovirus vectors” as used herein include any and all adenoviruses or adenovirus vectors which have one or more endogenous genes or gene fragments deleted from it. In contrast, the terms “fully deleted adenovirus” and “fully deleted adenovirus vector” as used herein include any and all adenoviruses and adenovirus vectors from which all endogenous adenoviral genes and genetic material are deleted with the exception of the internal terminal repeats (ITRs) and the packaging signal (Ψ). The term “adenoviral vector genome” as used herein includes the genetic material that is found in the adenovirus vector.

By “antigen” is meant a molecule, which contains one or more epitopes that will stimulate a host's immune system to make a cellular antigen-specific immune response, or a humoral antibody response. Thus, antigens include proteins, polypeptides, antigenic protein fragments, oligosaccharides, polysaccharides, and the like. Furthermore, the antigen can be derived from any known virus, bacterium, parasite, plants protozoans, or fungus, and can be a whole organism. The term also includes tumor antigens. Similarly, an oligonucleotide or polynucleotide which expresses an antigen, such as in DNA immunization applications, is also included in the definition of antigen. Synthetic antigens are also included, for example polyepitopes, flanking epitopes, and other recombinant or synthetically derived antigens (Bergmann et al. (1993) Eur. J. Immunol. 23:2777 2781; Bergmann et al. (1996) J. Immunol. 157:3242 3249; Suhrbier, A. (1997) Immunol. And Cell Biol. 75:402 408; Gardner et al. (1998) 12th World AIDS Conference, Geneva, Switzerland, Jun. 28-Jul. 3, 1998).

A “coding sequence” or a sequence which “encodes” a selected polypeptide, is a nucleic acid molecule which is transcribed (in the case of DNA) and translated (in the case of mRNA) into a polypeptide in vivo when placed under the control of appropriate regulatory sequences (or “control elements”). The boundaries of the coding sequence are determined by a start codon at the 5′ (amino) terminus and a translation stop codon at the 3′ (carboxy) terminus. A transcription termination sequence may be located 3′ to the coding sequence. Transcription and translation of coding sequences are typically regulated by “control elements,” including, but not limited to, transcription promoters, transcription enhancer elements, Shine and Delagamo sequences, transcription termination signals, polyadenylation sequences (located 3′ to the translation stop codon), sequences for optimization of initiation of translation (located 5′ to the coding sequence), and translation termination sequences.

The term “conserved antigens” as used herein refers to an antigen that shows similarities to gene variants expressed in a virus of more than one serotype.

The term “conservative variants” of amino acid residues as used herein reflects changes of amino acids in a given position in a protein with amino acids with similar biochemical properties, such as charge, hydrophobicity and size.

The term “construct” refers to at least one of a genetic composition or a composition in accordance with the present disclosure as either an adenovirus genome or as a packaging construct.

The term “delete” or “deleted” as used herein means expunging, erasing, or removing.

The terms “deleted Ad (virus) vector” and “gutted-,” “mini-,” “deleted-,” “.DELTA.,” or “pseudo-vectors,” as used herein, refer to a linear vector module with ITRs. These vectors can also code for some structural and/or nonstructural gene sequences and/or one or more genes of interest or transgenes.

The term “expression” refers to the transcription and/or translation of an endogenous gene, transgene or coding region in a cell.

A “ gene delivery vector,” “GDV,” “gene transfer vector,” or “gene transfer vehicle” is a composition including a packaged vector module of the present disclosure.

As used herein, the term “gene expression construct” refers to a promoter, at least a fragment of a gene of interest, and a polyadenylation signal sequence. A vector module of the present disclosure may comprise a gene expression construct.

The terms “gene of interest,” “GOI,” and “transgene,” as used herein, refer to genes that code for genes whose function is of medical interest and may not be a natural flavivirus gene. A gene of interest can be one that exerts its effect at the level of RNA or protein. Examples of genes of interest include, but are not limited to, therapeutic genes, immunomodulatory genes, virus genes, bacterial genes, protein production genes, inhibitory RNAs or proteins, and regulatory proteins.

A “gene sequence” refers to the order of nucleotides. A gene sequence can be regulatable. Regulation of gene expression can be accomplished by one of (1) alteration of gene structure: site-specific recombinases (e.g., Cre based on the Cre-loxP system) can activate gene expression by removing inserted sequences between the promoter and the gene; (2) changes in transcription: either by induction (covered) or by relief of inhibition; (3) changes in mRNA stability, by specific sequences incorporating in the mRNA or by siRNA; and (4) changes in translation, by sequences in the mRNA. Deleted adenoviruses are also called “high-capacity” adenoviruses. These deleted adenoviruses can accommodate up to 33 kb of genetic sequences.

The term “headless hemagglutinin” refers to a her agglutinin construct that consists of the hemagglutinin stem.

The terms “hemagglutinin stem” or “stem region” refer to a structural component of the influenza hemagglutinin protein that is relatively invariant and does not contain the genetic hypervariable region of the influenza hemagglutinin.

The term “heterologous” is used for any combination of DNA sequences that is not normally found intimately associated with nature.

The term “homology” refers to the existence of shared ancestry between a pair of structure or genes.

A “host cell” or “packaging cell” is a cell that is able to package adenovirus or adenovirus vector genomes or modified genomes to produce viral particles. It can be engineered to provide a missing gene product or its equivalent. Thus, packaging cells are able to package the adenovirus genomes into the adenovirus particle. The production of such particles requires that the genome be replicated and that those proteins necessary for assembling an infectious virus are produced. The particles also can require certain proteins necessary for the maturation of the viral particle. Such proteins can be provided by a vector, a packaging construct or by the packaging cell. Exemplary host cells (HCs) that may be used to make ap packaging cell line according to the present disclosure include, but are not limited to, A549, HeLa, MRC5, W138, CHO cells, Vero cells, human embryonic retinal cells, or any eukaryotic cells, as long as the host cells are permissive for growth of adenoviruses. Some host cell lines include adipocytes, chondrocytes, epithelial, fibroblasts, glioblastoma, hepatocytes, keratinocytes, leukemia, lympohoblastoid, monocytes, macrophages, myoblasts, and neurons. Other cell types include, but are not limited to, cells derived from primary cell cultures, e.g., human primary prostate cells, human embryonic retinal cells, human stein cells. Eukaryotic diploid and aneuploid cell lines are included within the scope of the disclosure. The packaging cell must be one that is capable of expressing the products of the different constructs described in here at the appropriate level for those products in order to generate a high titer stock of recombinant virus vectors.

An “immune response” is an acquired immune response, such as a cellular or humoral immune response.

In the context of the present disclosure, an “immunomodulatory molecule” is a polypeptide molecule that modulates, i.e., increase or decrease, a cellular and/or humoral host immune response directed to a target cell in an antigen-specific fashion, and preferably is one that decreases the host immune response. Generally, in accordance with the teachings of the present disclosure, the immunomodulatory molecule(s) will be associated with the target cell surface membrane, e.g., inserted into the cell surface membrane or covalently or non-covalently bound thereto, after expression from the GDVs described herein.

The terms “influenza virus,” “influenza virion,” and “influenza particle” as used herein include any and all viruses that may be categorized as an influenza virus, including any influenza virus that infects a human or an animal, including all groups, subgroups and serotypes.

The terms “introducing” or “transfection,” as used herein, refer to delivery of an expression vector to a host cell. A vector may be introduced into the cell by transfection, which typically means insertion of heterologous DNA or RNA into a cell by physical means (e.g., calcium phosphate transfection, electroporation, microinjection or lipofection); infection, which typically refers to introduction by way of an infectious agent, i.e., a virus; or transduction, which typically means stable infection of a cell with a virus or the transfer of genetic material from one microorganism to another by way of a viral agent (e.g., a bacteriophage). A vector may be a plasmid, virus or other vehicle.

The term “linear DNA” refers to non-circularized DNA molecules. The term “linear RNA” refers to non-circularized RNA molecules.

The term “naturally” as used herein refers to something as found in nature; wild type; innately or inherently.

The term “nucleic acid” refers to a deoxyribonucleotide or ribonucleotide polymer in either single- or double-stranded form, and unless otherwise limited, encompasses known analogues having the essential nature of natural nucleotides in that they hybridize to single-stranded nucleic acids in a manner similar to naturally occurring nucleotides (e.g., peptide nucleic acids). Nucleic acids are “operably linked” when placed into a functional relationship with another nucleic acid sequence. For example, a promoter or enhancer is operably linked to a coding sequence if it affects the transcription of the sequence. Generally, “operably linked” means that the DNA sequences being linked are contiguous. However, enhancers do not have to be contiguous. Linking is accomplished by ligation at convenient restriction sites. If such sites do not exist, the synthetic oligonucleotide adapters or linkers are used in accordance with conventional practice.

The term “non-structural genes,” as used herein refers to a group of genes present in the adenovirus genome. These genes are coding for “nonstructural genes.”

The term “packaging construct” or “packaging expression plasmid” refers to an engineered plasmid construct of circular, double-stranded DNA molecules, wherein the DNA molecules include at least a subset of adenovirus structural or nonstructural genes under control of a promoter. The “packaging construct” does not comprise more than one ITR or genetic information to enable independent virus replication to produce infections, viral particles and/or efficient packaging of this genetic material being packaged into a viral particle.

A cell that is “permissive” supports replication of a virus.

The term “plasmid,” as used herein, refers to an extra-chromosomal DNA molecule separate from the chromosomal DNA, which is capable of replication independently of the chromosomal DNA. In many cases, it is circular and double-stranded.

The term “polylinker” refers to a short stretch of artificially synthesized DNA, which carries a number of unique restriction sites allowing the easy insertion of any promoter or DNA segment.

The term “promoter” means a regulatory region of DNA that facilitates the transcription of a particular gene. Promoters usually comprises a TATA box capable of directing RNA polymerase II to initiate RNA synthesis at the appropriate transcription initiation site for a particular coding sequence. A promoter may additionally comprise other recognition sequences generally positioned upstream or 5′ to the TATA box, referred to as upstream promoter elements, which influence the transcription initiation rate. A “constitutive promoter” refers to a promoter that allows for continual transcription of its associated gene in many cell types. An “inducible-promoter system” refers to a system that uses a regulating agent (including small molecules such as tetracycline, peptide and steroid hormones, neurotransmitters, and environmental factors such as heat, and osmolarity) to induce or to silence a gene. Such systems are “analog” in the sense that their responses are graduated, being dependent on the concentration of the regulating agent. Also, such systems are reversible with the withdrawal of the regulating agent. Activity of these promoters is induced by the presence or absence of biotic or abiotic factors. Inducible promoters are a powerful tool in genetic engineering because the expression of genes operably linked to them can be turned on or off at certain stages of development of an organism or in a particular tissue.

The term “propagate” or “propagated” as used herein refers to the reproduction, multiplication, or other increase in number, amount, or extent by any process.

The term “purification” as used herein refers to the process of purifying, or freeing from substantially most, most, substantially all, or all foreign, extraneous, or objectionable elements.

A “regulatory sequence,” “regulatory region,” or “regulatory element” is a promoter, enhancer, or other segment of DNA where regulatory proteins such as transcription factors bind preferentially. They control gene expression and thus protein expression.

The term “recombinase” as used herein refers to an enzyme that catalyzes genetic recombination. A recombinase enzyme catalyzes the exchange of short pieces of DNA between two long DNA strands, particularly the exchange of homologous regions between the paired maternal and paternal chromosomes.

A “restriction enzyme” or “restriction endonuclease” is an enzyme that cuts double-stranded DNA.

The term “restriction site” or “restriction recognition site” refers to a particular sequence of nucleotides that is recognized by restriction enzymes as sites to cut the DNA molecule. The sites are generally, but not necessarily, palindromic (Because restriction enzymes usually bind as homodimers) and a particular enzyme may cut between two nucleotides within its recognition site, or somewhere nearby.

The term “replication” or “replicating” means making an identical copy of an object such as, for example, but not limited to, a virus particle.

As used herein the term “replication deficient” refers to the characteristic of a virus that is unable to replicate in a natural environment. A replication deficient virus is a virus that has been deleted of one or more of the genes that are essential for its replication, such as, for example, but not limited to, the E1 genes. Replication deficient viruses can be propagated in a laboratory in cell lines that express the deleted genes.

The terms “similarity” or “sequence similarity” as used herein refer to the measure of an empirical relationship between protein sequences. A similarity score as used herein describes an approximation of the evolutionary distance between and thus the similarity and/or identify of amino acid residues within a given protein.

The term “structural genes,” as used herein, refers to a group of genes present in the adenovirus genome that form the adenovirus capsid.

The term “stutter” refers to a DNA or RNA sequence that is inserted into another DNA or RNA sequence in order to increase its size. Stuffer fragments usually do not code for any protein nor contain regulatory elements for gene expression, such as transcriptional enhancers or promoters.

The term “target” or “targeted” as used herein refers to a biological entity, such as, for example, but not limited to, a protein, cell, organ, or nucleic acid, whose activity can be modified by an external stimulus. Depending upon the nature of the stimulus, there may be no direct change in the target, or a conformational change in the target may be induced.

The term “transfection” refers to the instruction into a cell genetic material as DNA or RNA (for example, introduction of an isolated nucleic acid molecule or a construct of the present disclosure). The term “transduction” as used herein refers to the introduction into a cell DNA either as DNA or by means of a GDV of the present disclosure. A GDV of the present disclosure can be transduced into a target cell.

The term “universal influenza vaccine” as used herein refers to an influenza vaccine that carries antigens that can induce immune responses in humans and animals against influenza viruses of more than one serotype or several influenza serotypes.

The term “untranslated region,” as used herein, refers to an RNA section that does not code for a protein.

The term “vector” refers to a nucleic acid used in infection of a host cell and into which can be inserted a polynucleotide. Vectors are frequently replicons. Expression vectors permit transcription of a nucleic acid inserted therein. Some common vectors include, but are not limited to, plasmids, cosmids, viruses, phages, recombinant expression cassettes, and transposons. The term “vector” may also refer to an element which aids in the transfer of a gene from one location to another.

The term “vector module” refers to an adenovirus genetic composition that is packaged in an adenovirus virion.

The term “viral DNA” or “viral RNA” as used herein refers to a sequence of DNA or RNA that is found in virus particle.

As used herein, a “viral genome” is the totality of the DNA or RNA that is found in virus particles, and that contain all the elements necessary for virus replication. The genome is replicated and transmitted to the virus progeny at each cycle of virus replication.

The term “virions” as used herein, refers to a viral particle. Each virion consists of genetic material within a protective protein capsid.

The term “wild-type” as used herein refers to the typical form of an organism, strain, gene, protein, nucleic acid, or characteristic as it occurs in nature. Wild-type refers to the most common phenotype in the natural population. The terms “wild-type” and “naturally occurring” are used interchangeably.

In accordance with embodiments of the present disclosure, a universal influenza vaccine is provided.

In an embodiment, the universal influenza vaccine is a multimeric vaccine comprising a set of antigens, the set of antigens containing at least two, and preferably more than two, different influenza hemagglutinin (HA)-derived antigens. Hemagglutinin is a glycoprotein found on the surface of influenza viruses and is integral to the viruses' infectivity.

An antigen derived from an influenza HA may be a DNA sequence or RNA sequence which encodes all or a portion of the influenza HA or a protein sequence for all or a portion of the influenza HA itself.

Influenza HAs have a hypervariable region located between conserved cysteines at positions 52 and 277, as numbered using the amino acid numbering of an influenza hemagglutinin of the serotype H3. In an embodiment, the DNA, RNA or protein sequence of the antigen which corresponds to the hypervariable region is deleted.

In an embodiment, the HA hypervariable region is replaced with a peptide linker (or DNA or RNA sequence corresponding to a peptide linker) to increase stability and cell surface expression of the headless hemagglutinin construct. An exemplary nonlimiting peptide linker is GGGGS-GGGGS-GGGGS-GGGGS (or (GGGGS)₄).

In an embodiment, the influenza HA derived antigens are based on headless influenza hemagglutinin proteins.

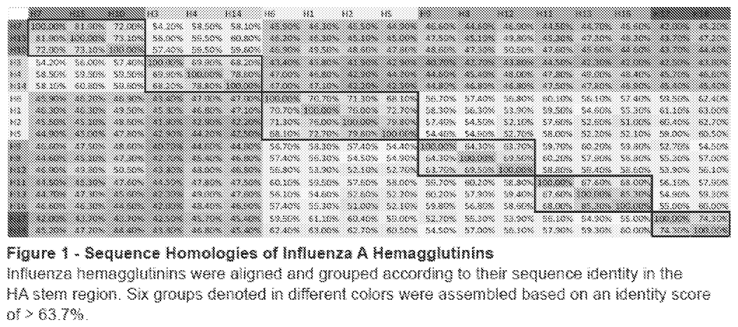

In an embodiment, the at least two, and preferably more than two, different influenza HA derived antigens are composed of sequences (whether DNA, RNA or protein) that have a similarity with HA sequences (DNA, RNA, or protein) of more than one influenza serotype in excess of a similarity score of 60, or 65, or 70, or 75, or 80, or 85, or 90, as calculated by the emboss explorer cons program. The similarity score is based on the presence of identical amino acid residues (or the respective coding DNA or RNA) at the corresponding location of the different HAs and the presence of conservative variants of amino acid residues (or the respective coding DNA or RNA) at the corresponding location of the different HAs. FIG. 1 provides visual illustrations of influenza HAs and the similarity scores by aligning and grouping the HAs according to the sequence identity in the HA stem region.

In an embodiment, representative hemagglutinins of certain serotypes of influenza A are used for the alignment of the different hemagglutinins. Exemplary representative influenza A serotypes include, but are not limited to:
H1: A/California148/2017 (H1N1)
H2: A/Moscow/1019/1965 (H2N2)
H3: A/Washington/16/2017 (H3N2) No 8
H4: A/duck/Guangdong/DGQTSJ147P/2015 (H4N8)
H5: A/Cygnus olor/Belgium/1567/2017 (H5N8)
H6: A/green-winged teal/Tennessee/17OS0651/2017 (H6N1)
H7: A/Guangdong/HP001/2017 (117N9)
H8: American black duck/Illinois/4119/2009 (H8N4)
H9: A/Japanese Quail/Vietnam/4/2009 (H9N2)
H10: A/American black duck/Alberta/118/2016 (H10N7)
H11: A/duck/Memphis/546/1974
H12: American black duck/New Brunswick/00998/2010 (H1N6)
H13: American white pelican/Minnesota/Sg-0611/2008 (H13N9)
H14: A/Northern shoveler/Missouri/16OS6248/2016 (H14N7)
H15: A/duck/Bangladesh/24704/2015 (H15N9)
H16: A/glaucous-0winged gull/Southcentral Alaska/16MB03160/2016 (H16N3)
H17: A|H17N10|09/2010|A/little_yellow_shouldered_bat/Guatemala/060/2010
H18: 4|HA|A|A/dark_fruit_eating_bat/Bolivia/PBV780_781/2011

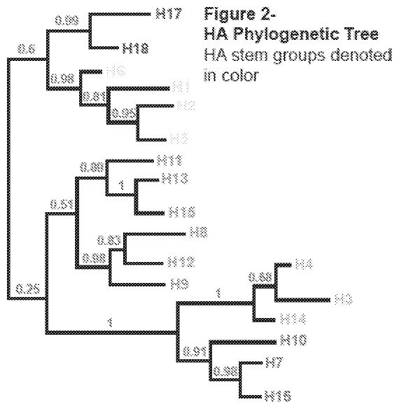

In another embodiment, the similarity score, as used herein, describes an approximation of the evolutionary distance between (and thus, the similarity and/or identify of amino acid residues within) given proteins. Based on that data, a family tree of the different hemagglutinin serotypes is created that depicts the evolutionary connection and distance of the different hemagglutinins as a phylogenetic tree (FIG. 2).

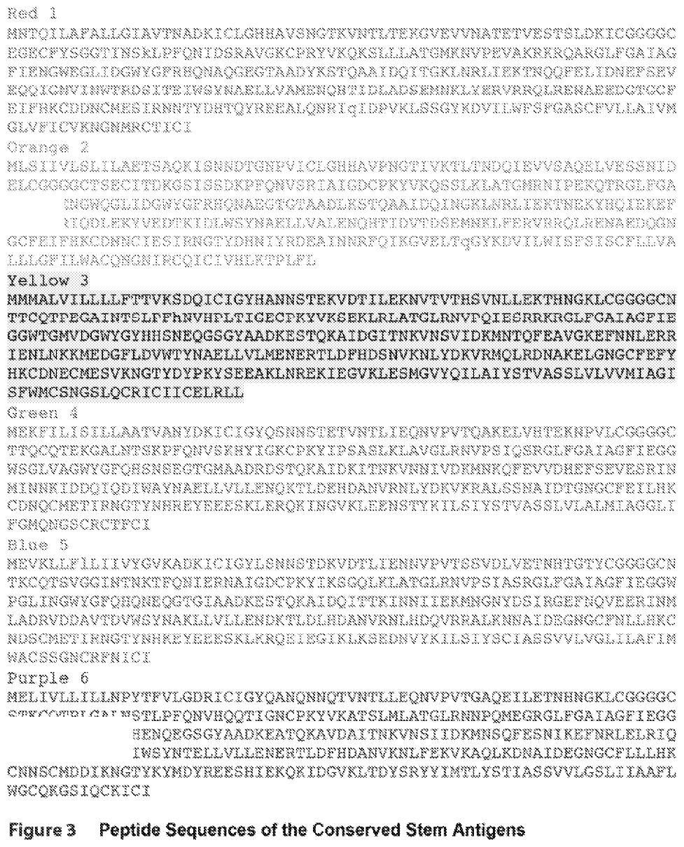

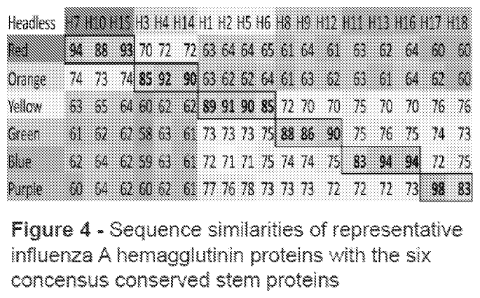

In another embodiment, several conserved headless hemagglutinin protein constructs are designed based on these similarity scores. Exemplary peptide sequences of conserved constructs are shown in FIG. 3, with the sequence similarities of these sequences relative to influenza A hemagglutinins shown in FIG. 4.

As set forth herein, a universal influenza virus vaccine includes a set of antigens composed of at least two, or preferably more than two, different influenza hemagglutinin (HA) derived antigens. In an embodiment, the at least two, and preferably more than two, different influenza HA derived antigens are at least two, and preferably more than two, of the conserved headless hemagglutinin protein constructs chosen from the sequences provided in FIG. 3 or chosen from the sequences conHA1, conCHA2, conHA3, conH4, conH5 and conH6. In an embodiment, the at least two, or preferably more than two, antigens are provided to humans and/or animals in the form of a vaccine.

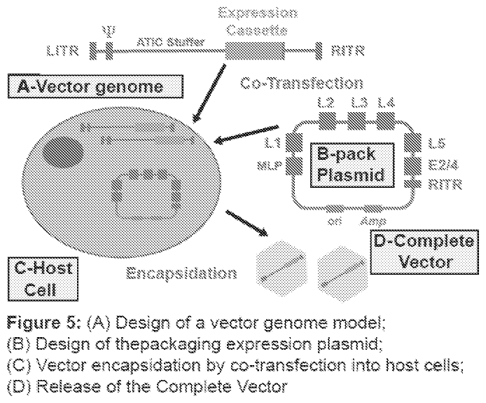

In an embodiment, the at least two, or preferably more than two, influenza HA derived antigens are provided as proteins, combined to virus like particles, delivered in nanoparticles, delivered in emulsion, their sequences integrated as transgenes into DNA or RNA vaccines, or their sequences integrated into a viral vector. FIG. 5 is a schematic representation of a viral vector model.

In another embodiment, vaccines of the present disclosure deliver the influenza HA derived antigens in a suitable pharmaceutical carrier formulated using standard methods of vaccine formulation in the presence or absence of immune enhancing moieties, such as, but not limited to, vaccine adjuvants and interleukins.

In another embodiment, one or more of the influenza HA derived antigen constructs are delivered as vaccines to human and animals through different application routes, including but not limited to intramuscular, intranasal, intracutaneous, subcutaneous, oral, intrarectal or intrabronchiolar.

In another embodiment, one or more of the influenza HA derived antigen constructs are delivered as transgene constructs in an adenoviral vector.

In another embodiment, one or more of the influenza HA derived antigen constructs are delivered as transgene constructs in a fully deleted adenoviral vector, such as, but not limited to, a fully deleted helper virus-independent adenoviral vector.

In another embodiment, one or more of the influenza HA derived antigen constructs contain a promoter and a polyadenylation site. In another embodiment, transgene constructs are linked by internal ribosomal entry sites. In another embodiment, transgene constructs code for more than one influenza HA derived antigen construct linked by internal ribosomal entry sites. In another embodiment, transgene constructs code for more than one influenza HA derived antigen construct linked by linker self-digesting enzymes or digestible by protein digesting enzymes.

In another embodiment, transgene constructs of the influenza HA derived antigen constructs are coded within vectors, such as, but not limited to, adenoviral vectors as replication efficient RNAs, such as, but not limited to, an alpha virus replicon.

In an embodiment, a method of producing a universal influenza vaccine is provided. In embodiments, the method includes obtaining at least two, and preferably more than two, different influenza HA derived antigens having a similarity with HA molecules of more than one influenza serotype in excess of a similarity score of at least 60, or 65, or 70, or 75, or 80, or 85, or 90. The at least two, and preferably more than two, different influenza HA derived antigens are then provided as DNA sequences the code for the HA antigen, RNA sequences that code for the HA antigen, or protein sequences for inclusion in a vaccine composition. The at least two, and preferably more than two, influenza HA derived antigens may be in accordance with any embodiment or combination of embodiments provided herein. The at least two, and preferably more than two, different influenza HA derived antigens may be provided in the form of a viral vector for inclusion in a vaccine composition.

In an embodiment, a method of vaccinating an animal (including, for example, humans) against at least two, and preferably more than two, influenza A subtypes comprises providing a vaccine composition comprising at least two, and preferably more than two, different influenza HA derived protein antigens.

### EXAMPLE

### Example 1—Design of Influenza HA Derived Antigens

Protein Sequences of the examples of protein sequences of influenza HAs of different serotypes are aligned. Protein sequences used in the examples are from the following influenza A subtypes:
H1: A/California/48/2017 (H1N1)
H2: A/Moscow/1019/1965 (H2N2)
H3: A/Washington/16/2017 (H3N2) No 8
H4: A/duck/Guangdong/DGQTSJ147P/2015 (H4N8)
H5: A/Cygnus olor/Belgium/1567/2017 (H5N8)
H6: A/green-winged teal/Tennessee/17OS0651/2017 (H6N1)
H7: A/Guangdong/HP001/2017 (H7N9)
H8: American black duck/Illinois/4119/2009 (H8N4)
H9: A/Japanese Quail/Vietnam/4/2009 (H9N2)
H10: A/American black duck/Alberta/118/2016 (H10N7)
H11: A/duck/Memphis/546/1974
H12: American black duck/New Brunswick/00998/2010 (H12N6)
H13: American white pelican/Minnesota/Sg-0611/2008 (H13N9)
H14: A/Northern shoveler/Missouri/16OS6248/2016 (H14N7)
H15: A/duck/Bangladesh/24704/2015 (H15N9)
H16: A/glaucous-0winged gull/Southcentral Alaska/16MB03160/2016 (H16N3)
H17: A|H17N10|09/2010|A/little_yellow_shouldered_bat/Guatemala/060/2010
H18: 4|HA|A|A/dark_fruit_eating_bat/Bolivia/PBV780_781/2011

Based on the analysis of the similarities of the different HA serotypes, the serotypes are grouped into six similarity groups, as depicted in FIGS. 1 and 2. HA sequences are downloaded from fludg.org. Incomplete sequences are removed for analysis. The remaining sequences are aligned using the MAFFT online server version 7: https://mafft.cbrc.jp/alignment/server/large.html?aug31. The following settings are used for analysis:
i. FFT-NS-2
ii. Memory usage: normal
iii. Scoring matrix for amino acid sequences: BLOSUM62
iv. Gap opening penalty: 5

Based in this alignment, the HA serotypes are grouped into the following groups:
H-I (red): with HA serotypes H7, H10, H15
H-II (orange) with HA serotypes H13, H14, H14
H-III (yellow) with HA serotypes H1, H2, H5, H6
H-IV (green) with HA serotypes H8, H9, H12
H-V (blue) with HA serotypes H11, H13, H15
H-VI (purple) with HA serotypes H17, H18

With these alignments, the rates of identity of the amino acid residues within the entire HA protein of each of the different HA groups exceeds a value of 63.7% identity for the HA proteins analyzed, as depicted in FIG. 1.

### Example 2—Sequence of Influenza HA Derived Antigens

Based on the analysis of Example 1, consensus proteins of the different HA serotypes are developed for the conserved regions of the HAs. The consensus sequences for each HA serotype group H1 through H18 are derived with emboss explorer cons: http://www.bioinformatics.nl/cgibin/emboss/cons. The following settings were used:
i. Plurality check set to 1
ii. Required number of identities at position: 1

In step 1, consensus sequences for each of the eighteen HA serotypes is developed. The sequences are found in fludb.org.

The following number of sequences are used to derive the respective HA serotype consensus sequences:
H1: 22868
H2: 622
H3: 25600
H4: 1932
H5: 5694
H6: 1745
H7: 2743
H8: 150
H9: 3885
H10: 1236
H11: 670
H12: 210
H13: 6856
H14: 31
H15: 21
H16: 268
H17: 3
H18: 2

Step 1: Once the HA consensus sequences are identified, the headless version of the consensus sequences for each of the six groups is determined in the following way:
(a) The 18 representative consensus sequences are aligned with clustal W in bioedit
(b) The universally conserved cysteine is identified at position 60 as the residue immediately before deletion
(c) The universally conserved CxxxC at around position 287-290 (around 100 aa from the universally conserved GLFGAIA sequence) is identified and used as the end of the headless deletion
(d) The region between the two cysteines in (c) is deleted and replaced with GGGG or (GGGGS)ₙ with n≥1

In FIG. 3, the size protein consensus sequences are listed with a monomer peptide linker.

### Example 3—Similarity of the Conserved HA Region Consensus Sequences

The six protein consensus sequences shown in FIG. 3 are aligned with the HA protein sequences of the HA serotypes assigned to each of the consensus sequences. A rate of similarity is calculated that takes into consideration amino acid identity as well as similarity of conservative amino acid changes in an amino acid residue in a given position. The scores are calculated with the emboss explorer cons program and provided in FIG. 4. Notably, the scores exceed 83 in all cases.

### Example 4—Design and Construction of Fully Deleted Ad Viral Vector Carrying Transgenes for Six Conserved Universal Influenza HA Derived Antigen Constructs

A fdAd vector is deleted of all endogenous ad genes. The space in the Ad genome can accommodate transgene constructs of a length of up to 33 kb. A fdAd vector genome only carries adenoviral sequences that correspond to the adenoviral inverted terminal repeats (ITR) and an Ad packaging signal, such as the adenoviral Ψ.

A fdAd viral vector genome is packaged into an adenoviral capsid in a host or packaging cell that is provided with the deleted adenoviral genes necessary for the genome necessary for encapsidation by a second genetic construct. That second genetic construct can be provided by, but not limited to, a helper virus construct or a packaging expression plasmid.

In an example, the fdAd vector genome once linearized is co-transfected with a packaging expression plasmid carrying genes coding for an Ad capsid of the human serotype 6 into host cells, such as, but not limited to cells of the human embryonic kidney cell line HEK-293 as depicted in FIG. 5. The encapsidated adenoviral vectors once released from the cells are purified and used, for example, for vaccination.

In an example, a fdAd carries transgene constructs coding for the six consensus sequences. Three transgene expression cassettes are produced with the following composition:
No. 1: a cytomegalovirus promoter enhancer sequence followed by, a transgene coding for the H-I (red) consensus protein followed by an internal ribosomal entry site followed by a transgene coding for the H-II (orange) consensus protein followed by a polyadenylation site.
No. 2: a cytomegalovirus promoter enhancer sequence followed by a transgene coding for the H-III (yellow) consensus protein followed by an internal ribosomal entry site followed by a transgene coding for the H-IV (green) consensus protein followed by a polyadenylation site.
No. 3: a cytomegalovirus promoter enhancer sequence followed by a transgene coding for the H-V (blue) consensus protein followed by an internal ribosomal entry site followed by a transgene coding for the H-VI (purple) consensus protein followed by a polyadenylation site.

The three transgene constructs, Nos. 1, 2 and 3 are moved into fully deleted adenoviral vector genome.

### Example 5—Protocol to Immunize Humans or Animals with a fdAd Vector C Transgenes for Six Conserved Universal Headless HA Protein Constructs

The fdAd vector carrying the transgenes for the six consensus conserved HA proteins is encapsidated into an Ad transcript as described in Example 4. It is suspended in a physiological solution and used to immunize humans and/or animals. For this purpose, it is delivered to the recipient by injection via, but not limited to, the intramuscular, intradermal, subcutaneous route or by other routes such as, hut not limited to, intranasal or oral routes.

Both humoral and cellular immune responses are induced in humans and animals that have been exposed to this adenoviral vector. These immune responses protect humans and animals against a challenge of an influenza virus that belongs to a member of any influenza A serotype.

While multiple embodiments of a universal influenza vaccine and related methods have been described in detail herein, it should be apparent that modifications and variations thereto are possible, all of which fall within the true spirit and scope of the invention. Further, since numerous modifications and changes will readily occur to those skilled in the art, it is not desired to limit the invention to the exact construction and operation shown and described, and accordingly, all suitable modifications and equivalents may be resorted to, falling within the scope of this disclosure.

1. A composition for a vaccine comprising:
at least two different influenza hemagglutinin (HA) derived antigens, wherein an HA protein from which the antigens are derived includes a hypervariable region and the hypervariable region is deleted from the at least different influenza HA derived antigens, and wherein each of the at least two different influenza HA derived antigens has a similarity with HA molecules of more than one influenza serotype in excess of at least 60, as calculated by the emboss explorer cons program.

2. The composition of claim 1, wherein the similarity is at least 70.

3. The composition of claim 1, wherein the similarity is at least 80.

4. The composition of claim 1, wherein the hypervariable region is replaced by a peptide linker in the at least two different influenza HA derived antigens.

5. The composition of claim 1, wherein the at least two different influenza HA derived antigens are proteins.

6. The composition of claim 1, wherein the at least two different influenza HA derived antigens are one of RNA and DNA coding for the HA protein from which the antigens are derived.

7. The composition of claim 6, wherein the one of RNA and DNA are in a viral vector.

8. The composition of claim 1, wherein the hypervariable region is located 20 between conserved cysteines at positions 52 and 277 using the amino acid numbering of an influenza HA of the serotype H3.

9. The composition of claim 1, comprising more than two different influenza HA derived antigens.

10. A method for producing a universal influenza vims vaccine comprising:
obtaining a first influenza HA derived antigen having a similarity with HA molecules of a first plurality of influenza serotypes in excess of at least 60, as calculated by the emboss explorer cons program; and
obtaining a second influenza HA derived antigen having a similarity with HA molecules of a second plurality of influenza serotypes in excess of at least 60, as calculated by the emboss 30 explorer cons program,
wherein the first and second plurality of influenza serotypes are composed of different serotypes.

11. The method of claim 10, wherein the first and second plurality of influenza serotypes are composed of different and non-overlapping serotypes.

12. The method of claim 10, where the HA proteins from which the first and second influenza HA derived antigens are derived contains a hypervariable region, and the hypervariable region is deleted from the first and second influenza HA derived antigens.

13. The method of claim 10, wherein the similarities are, independently, at least 70.

14. The method of claim 10, wherein the similarities are, independently, at least 80.

15. The method of claim 10, wherein the hypervariable region is replaced by a peptide linker.

16. The method of claim 10, wherein the first and second influenza HA derived antigens are proteins.

17. The method of claim 10, wherein the first and second influenza HA derived antigens are one of RNA and DNA coding for the HA protein from which the antigens are derived.

18. The method of claim 17, comprising encapsidating the one of the RNA and DNA coding the HA protein into a viral vector.

19. The method of claim 10, comprising obtaining at least a third influenza HA derived antigen having a similarity with HA molecules of a third plurality of influenza serotypes in excess of at least 60, as calculated by the emboss explorer cons program.

20. A method of vaccinating an animal against at least two different influenza serotypes comprising:
providing a vaccine composition comprising at least two different influenza hemagglutinin (HA) derived antigens, wherein an HA protein from which the antigens are derived includes a hypervariable region and the hypervariable region is deleted from the at least two different influenza HA derived antigens, and wherein each of the at least two different influenza HA derived antigens has a similarity with HA molecules of more than one influenza serotype in excess of at least 60, as calculated by the emboss explorer cons program: and
delivering the vaccine composition to the animal.
